(19) United States
(12) Patent Application Publication
(10) Pub. No.: US 2005/0075271 A1
(43) Pub. Date: Apr. 7, 2005

(54) Crystalline beta2 adrenergic receptor agonist

(76) Inventors: Martin S. Linsell, San Francisco, CA (US); John R. Jacobsen, San Mateo, CA (US); Davar Khossravi, San Mateo, CA (US); Mehdi Paborji, Cupertino, CA (US); Weijiang Zhang, Cupertino, CA (US)

Correspondence Address: THERAVANCE, INC., 901 GATEWAY BOULEVARD, SOUTH SAN FRANCISCO, CA, 94080, US

(21) Appl. No.: 10/627,555

(22) Filed: Jul. 25, 2003

Related U.S. Application Data
(60) Provisional application No. 60/398,678, filed on Jul. 26, 2002.
(60) Provisional application No. 60/398,928, filed on Jul. 26, 2002.

(51) Int. Cl. A61K 31/00

(52) U.S. Cl. 514/1

(57) ABSTRACT

The invention provides a crystalline salt form of a novel β₂ adrenergic receptor agonist. The invention also provides pharmaceutical compositions comprising the crystalline form, formulations containing the pharmaceutical compositions, methods of using the crystalline salt form to treat diseases associated with β₂ adrenergic receptor activity, and processes useful for preparing such a crystalline compound.

### CROSS REFERENCE TO RELATED APPLICATION

This application claims the benefit of U.S. Provisional Application Nos. 60/398,678 and 60/398,928, filed Jul. 26, 2002, the entire disclosures of which are incorporated herein by reference.

### FIELD OF THE INVENTION

The invention is directed to a crystalline form of a β₂ adrenergic receptor agonist. The invention is also directed to pharmaceutical compositions comprising the crystalline agent, formulations containing the pharmaceutical compositions, methods of using the crystalline agent to treat diseases associated with β₂ adrenergic receptor activity, and processes useful for preparing such a crystalline compound.

### BACKGROUND OF THE INVENTION

β₂ Adrenergic receptor agonists are recognized as effective drugs for the treatment of pulmonary diseases such as asthma and chronic obstructive pulmonary disease (including chronic bronchitis and emphysema). β₂ Adrenergic receptor agonists are also useful for treating pre-term labor, and are potentially useful for treating neurological disorders and cardiac disorders. Commonly assigned U.S. Pat. No. 6,576,793 B1discloses the novel compound N-{2-[4-((R)-2-hydroxy-2-phenylethylamino)phenyl]ethyl}-(R)-2-hydroxy-2-(3-formamido-4-hydroxyphenyl)ethylamine, 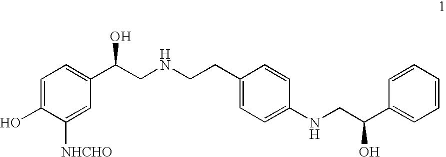
as a potent β₂ adrenergic receptor agonist. Compound 1 is alternatively referenced by the chemical names N-[3-[(1R)-1-hydroxy-2-[[2-[4-[((2R)-2-hydroxy-2-phenylethyl)amino]phenyl]ethyl]amino]ethyl-6-hydroxyphenyl]-formamide and (α-R)-3-formamido-4-hydroxy-α-[[[p-(N-((2R)-hydroxy-phenethyl))-amino-phenethyl]amino]methyl benzyl alcohol.

Active agents for the treatment of pulmonary diseases are advantageously administered by inhalation. Inhalation is an effective means for delivering an agent directly to the respiratory tract. There are three general types of pharmaceutical inhalation devices: nebulizer inhalers, dry powder inhalers (DPI), and metered-dose inhalers (MDI). Preparation of formulations for administration by inhalation typically relies on the existence of a crystalline form of the active agent, or of a crystalline form of a pharmaceutically acceptable salt of the active agent, having suitable physical and chemical properties. For example, it is desirable that crystalline salts used in dry powder and suspension formulations administered by inhalation typically be non-hygroscopic and stable upon micronization.

It is also desirable for solution formulations of such agents to be stable upon long term storage. However, for the case of the β₂ adrenergic receptor agonist formoterol tartrate, stability of a solution formulation for nebulizer administration has been identified as a limitation to its acceptability. As reported in U.S. Pat. No. 6,040,344, “because of the problematic stability of R,R-formoterol L-tartrate in aqueous solution, this formulation is not attractive for long term storage.”

No crystalline form of compound 1 or of a salt thereof, nor of a formulation comprising compound 1 that is suitable for adminstration by inhalation has been reported previously.

### SUMMARY OF THE INVENTION

The present invention provides a crystalline form of N-{2-[4-((R)-2-hydroxy-2-phenylethylamino)phenyl]ethyl}-(R)-2-hydroxy-2-(3-formamido-4-hydroxyphenyl)ethylamine dihydrochloride. The dihydrochloride salt of compound 1 has been characterized by x-ray powder diffraction (XRPD), differential scanning calorimetry (DSC), infrared spectroscopy (IR), nuclear magnetic resonance spectroscopy (NMR), and by elemental analysis.

Surprisingly, the crystalline dihydrochloride salt of compound 1 can be ground into micronized particles without significant decomposition. Additionally, the crystalline dihydrochloride salt has been found to be neither hygroscopic nor deliquescent, even when exposed to the atmosphere for prolonged periods at high relative humidity; and to be thermally stable at elevated temperatures.

The invention also provides pharmaceutical compositions comprising the dihydrochloride salt of compound 1 and a pharmaceutically acceptable carrier. The pharmaceutical compositions include formulations that are specifically prepared for administration by inhalation.

In particular, the present invention provides an aqueous pharmacuetical composition suitable for nebulizer administration. The present aqueous aerosol formulation comprises N-{2-[4-((R)-2-hydroxy-2-phenylethylamino)phenyl]ethyl}-(R)-2-hydroxy-2-(3-formamido-4-hydroxyphenyl)ethylamine dihydrochloride, a buffering agent, and water. The formulation is buffered to a pH of between about 4 and about 6, preferably between about 5 and about 5.5, and more preferably about 5, and desirably is isotonic. The formulation can be adjusted to isotonicity by the addition of a suitable salt, for example, sodium chloride. Optionally, the formulation can also include a surfactant.

Further, the invention provides combinations comprising the crystalline dihydrochloride salt of compound 1 and one or more other therapeutic agents and pharmaceutical compositions comprising such combinations.

In another aspect, the invention provides a method of treating a disease or condition associated with β₂ adrenergic receptor activity (e.g. a pulmonary disease, such as asthma or chronic obstructive pulmonary disease, pre-term labor, a neurological disorder, a cardiac disorder, or inflammation) in a mammal, the method comprising administering to the mammal, a therapeutically effective amount of the crystalline dihydrochloride salt of compound 1. The invention further provides a method of treatment comprising administering a combination of a a therapeutically effective amount of the crystalline dihydrochloride salt of compound 1 together with one or more other therapeutic agents.

In yet another aspect, the invention provides a process for preparing the crystalline dihydrochloride salt of compound 1, comprising the steps of dissolving N-{2-[4-((R)-2-hydroxy-2-phenylethylamino)phenyl]ethyl}-(R)-2-hydroxy-2-(3-formamido-4-hydroxyphenyl)ethylamine in a polar solvent to form a first solution; and adding hydrochloric acid to form a second solution from which the crystalline salt of the invention is formed. The invention further provides a crystalline hydrochloride salt produced by the above process. Optionally, the process also includes a subsequent recrystallization step comprising dissolving the crystalline diHCl salt in a polar solvent, optionally adding between about 0.5 and about 1.5 equivalents of hydrocholoric acid per mole of free base, and adding a polar solvent to form a solution from which the crystalline salt of the invention is formed.

The invention further provides a process for preparing the intermediate 2-[4-((R)-2-hydroxy-2-phenylethylamino)phenyl]ethylamine (2) which is useful for preparing compound 1. The intermediate is formed by reacting 2-(4-aminophenyl)ethylamine or a salt thereof with a sufficient amount of base to substantially deprotonate the 4-amino group; and reacting the resulting product with (R)-styrene oxide to provide intermediate 2.

### BRIEF DESCRIPTION OF THE DRAWINGS

The invention is illustrated by reference to the accompanying drawings.

FIG. 1 shows an x-ray powder diffraction pattern of N-{2-[4-((R)-2-hydroxy-2-phenylethylamino)phenyl]ethyl}-(R)-2-hydroxy-2-(3-formamido-4-hydroxyphenyl)ethylamine dihydrochloride.

FIG. 2 shows a differential scanning calorimetry trace of N-{2-[4-((R)-2-hydroxy-2-phenylethylamino)phenyl]ethyl}-(R)-2-hydroxy-2-(3-formamido-4-hydroxyphenyl)ethylamine dihydrochloride.

### DETAILED DESCRIPTION OF THE INVENTION

When describing the compounds, compositions and methods of the invention, the following terms have the following meanings, unless otherwise indicated.

The term “therapeutically effective amount” refers to an amount sufficient to effect treatment when administered to a patient in need of treatment.

The term “treatment” as used herein refers to the treatment of a disease or medical condition in a patient, such as a mammal (particularly a human) which includes:
(a) preventing the disease or medical condition from occurring, i.e., prophylactic treatment of a patient;
(b) ameliorating the disease or medical condition, i.e., eliminating or causing regression of the disease or medical condition in a patient;
(c) suppressing the disease or medical condition, i.e., slowing or arresting the development of the disease or medical condition in a patient; or
(d) alleviating the symptoms of the disease or medical condition in a patient.

The phrase “disease or condition associated with β₂ adrenergic receptor activity” includes all disease states and/or conditions that are acknowledged now, or that are found in the future, to be associated with β₂ adrenergic receptor activity. Such disease states include, but are not limited to, bronchoconstrictive or pulmonary diseases, such as asthma and chronic obstructive pulmonary disease (including chronic bronchitis and emphysema), as well as neurological disorders and cardiac disorders. β₂ Adrenergic receptor activity is also known to be associated with pre-term labor (see U.S. Pat. No. 5,872,126) and some types of inflammation (see WO 99/30703 and U.S. Pat. No. 5,290,815).

The term “isotonic” as used herein means having an osmotic pressure equal or similar to that of physiological fluids. Body fluids normally have an osmotic pressure that often is described as corresponding to that of a 0.9% (w/v) aqueous solution of sodium chloride.

The term “buffer” or “buffered” as used herein refers to a solution containing both a weak acid and its conjugate base, whose pH changes only slightly upon addition of acid or base. The term “buffering agent” refers to a species whose inclusion in a solution provides a buffered solution.

The present invention provides a crystalline form of N-{2-[4-((R)-2-hydroxy-2-phenylethylamino)phenyl]ethyl}-(R)-2-hydroxy-2-(3-formamido-4-hydroxyphenyl)ethylamine dihydrochloride. As used herein the terms “dihydrochloride” and “diHCl salt of the present invention” refer to a material having between about 1.65 and about 2.10 equivalents of chlorine, preferably, between about 1.90 and about 2.05 equivalents of chlorine per mole of free base material.

The crystalline form of the present invention is characterized by an x-ray powder diffraction pattern having two or more diffraction peaks at 2θ values selected from the group consisting of 15.61±0.2, 16.32±0.2, 19.50±0.2, 24.25±0.2, 24.92±0.2, 25.45±0.2, 28.67±0.2, and 31.16±0.2. In particular, the present crystalline form is characterized by an x-ray powder diffraction pattern comprising diffraction peaks at 20 values of 24.25±0.2, 24.92±0.2, 25.45±0.2.

The invention further provides a crystalline hydrochloride salt of N-{2-[4-((R)-2-hydroxy-2-phenylethylamino)phenyl]ethyl}-(R)-2-hydroxy-2-(3-formamido-4-hydroxyphenyl)ethylamine characterized by an x-ray powder diffraction pattern having two or more diffraction peaks at 20 values selected from the group consisting of 15.61±0.2, 16.32±0.2, 19.50±0.2, 24.25±0.2, 24.92±0.2, 25.45±0.2, 28.67±0.2, and 31.16±0.2.

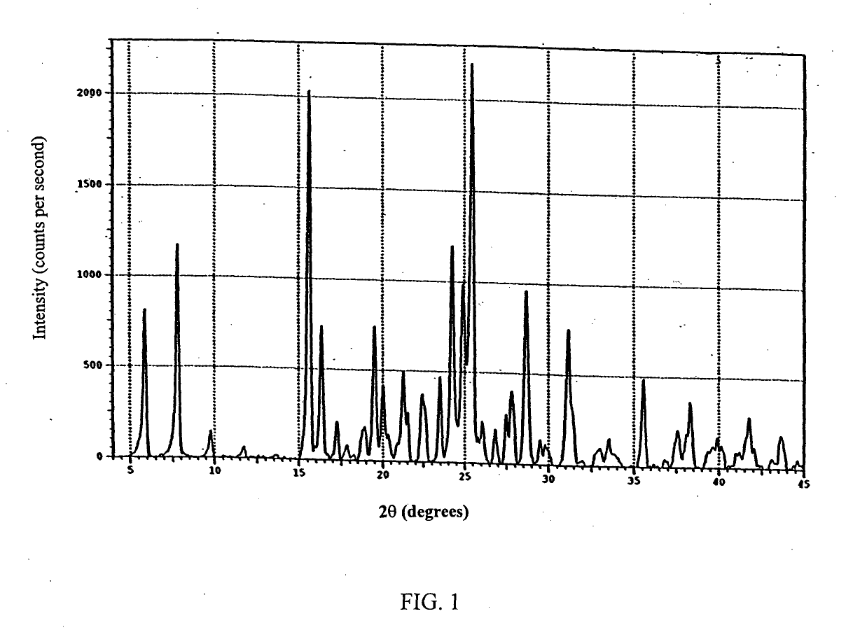

As is well known in the field of x-ray powder diffraction, relative peak heights of XRPD spectra are dependent on a number of factors having to do with sample preparation and instrument geometry, while peak positions are relatively insensitive to experimental details. Thus, the crystalline diHCl salt of compound 1 is also characterized by an x-ray powder diffraction pattern in which the peak positions are substantially in accordance with those shown in FIG. 1.

The crystalline form of the present diHCl salt is further characterized by its infrared absorption spectrum which shows significant absorption bands at 696±1, 752±1, 787±1, 827±1, 873±1, 970±1, 986±1, 1020±1, 1055±1, 1066±1, 1101+1, 1197±1, 1293±1, 1371±1, 1440±1, 1542±1, 1597±1, 1658±1, 2952±1, 3372±1, and 3555±1 cm⁻¹.

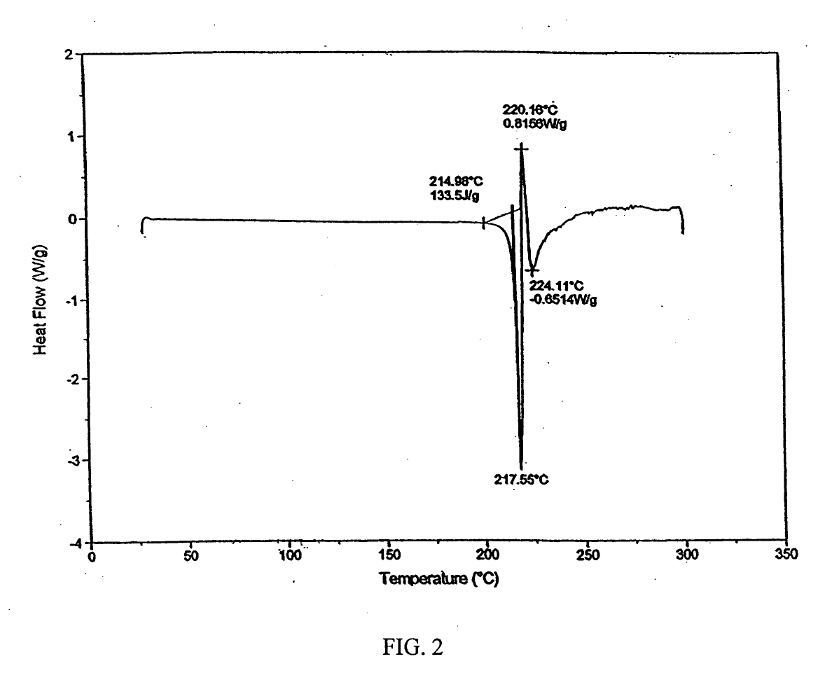

The crystalline diHCl salt of the present invention is yet further characterized by its differential scanning calorimetry trace which shows an onset of endothermic heat flow at about 200° C., as illustrated in FIG. 2. Without being bound to any theory of action, comparison of the DSC trace with thermogravimetric analysis data supports the inference that the crystal form of the present invention exhibits simultaneous melting and decomposition as the temperature is scanned above the onset temperature of about 200° C.

The present crystalline diHCl salt has been demonstrated to be stable upon exposure to elevated temperature and humidity. For example, after storage for 30 days at 40° C. and 75% humidity, analysis by DSC shows no detectable difference and analysis by high pressure liquid chromatography (HPLC) shows no appreciable chemical degradation of either crystalline material in the form obtained directly by crystallization or of material that has been ground. In another test, the chemical purity of the present crystalline material was essentially unchanged after storage for 6 months at 40° C. and 75% humidity.

The present material can be provided as particles having a size of between about 1 and about 10 μm, which is generally accepted as the particle size range appropriate for administration by inhalation. Thus the present crystalline diHCl salt of compound 1 is suitable for preparation of pharmaceutical compositions, in particular, pharmaceutical compositions formulated for administration by inhalation.

The crystalline dihydrochloride salt of the present invention can be formed by the addition of at least two equivalents of hydrochloric acid to the active compound 1 dissolved in a polar solvent. To induce crystallization, preferably the solution from which the diHCl product is crystallized includes isopropanol and water in a ratio of isopropanol to water of from about 4:1 to about 10:1, volume to volume. More preferably, the ratio of isopropanol to water is from about 4:1 to about 7:1, volume to volume. The water component can be present in the polar solvent or can be introduced as aqueous hydrochloric acid. Preferably the ratio of total solvent to free base material is from about 15:1 to about 50:1, volume (mL) to weight (g). Optionally, the mixture of compound 1 and polar solvent can be heated to dissolve the freebase and the resulting mixture cooled back to room temperature before addition of hydrochloric acid.

For example, the crystalline diHCl salt can be formed by dissolving compound 1 in isopropanol at a temperature of between about 40° C. and about 60° C., cooling to room temperature, adding aqueous hydrochloric acid, and stirring during crystallization. The crystalline product can be isolated by filtration and dried under vacuum. In one aspect, therefore, the invention provides the crystalline hydrochloride salt produced by the process described above.

Optionally, the crystalline diHCl salt can be recrystallized by redissolving the crystalline salt in a polar solvent as described above. To ensure that the recrystallized product has two equivalents of HCl per mole of free base, particularly when preparing the product at greater than gram scale, hydrochloric acid can be included in the polar solvent. In these preparations, between about 0.5 and about 1.5, for, example, about 1 equivalent of HCl per mole of free base is usefully included in the polar solvent.

As illustrated in Examples 7a and 7b below, the crystalline diHCl salt can be recrystallized by dissolving in a mixture of isopropanol and water at elevated temperature, optionally adding hydrochloric acid, diluting with isopropanol, and cooling to room temperature with stirring. The product can be isolated by filtration and dried under vacuum.

The present invention also provides an aqueous pharmaceutical composition comprising N-{2-[4-((R)-2-hydroxy-2-phenylethylamino)phenyl]ethyl}-(R)-2-hydroxy-2-(3-formamido-4-hydroxyphenyl)ethylamine dihydrochloride, hereinafter “active salt,” that is suitable for nebulizer administration. The aqueous pharmaceutical composition alternatively can be administered by intramuscular injection or by intravenous injection. The active salt is preferably provided in a crystalline form as the crystalline diHCl salt described above. The present aerosol formulation comprises the active salt in an aqueous solution that is buffered to a pH of between about 4 and about 6, preferably between about 5 and about 5.5, and more preferably about 5.

The formulation according to the present invention contains between about 0.06 μg and about 1.2 mg, preferably between about 0.29 μg and about 234 μg of N-{2 -[4-((R)-2-hydroxy-2-phenylethylamino)phenyl]ethyl}-(R)-2-hydroxy-2-(3-formamido-4-hydroxyphenyl)ethylamine dihydrochloride per gram of solution.

The quantities of the dihydrochloride salt in the present formulation correspond to between about 0.05 μg and about 1 mg, preferably between about 0.25 μg and about 200 μg, of the free base active agent, compound 1, per gram of solution.

The aqueous pharmaceutical composition is formulated with a pharmaceutically acceptable buffering agent, which is present in solution in its protonated and its unprotonated form. The buffering agent is introduced as an acid or as the corresponding salt of the acid, preferably as the sodium salt, to maintain the pH of the solution in the specified range. Examples of suitable pharmaceutical buffering agents include citrate, phosphate, sulphate, acetate, succinate, maleate, and tartrate species. Citrate buffered solutions are preferred. The formulation is preferably adjusted to isotonicity with sodium chloride.

In addition, the formulation can optionally include up to about 1 mg per gram of solution of a pharmaceutically acceptable surfactant, such as polyethylene glycol sorbitan monooleate (Tween® 80), sorbitan trioleate (Span® 85), or the like. The formulation can be sterilized as appropriate.

Thus, suitable pharmaceutical formulations of the present invention consist of:
(a) 0.06 μg to 11.7 mg active salt
(b) 0.021 to 21 mg citric acid
(c) 0 to 11 mg NaCl
(d) 0 to 1 mg surfactant
(e) NaOH to adjust pH to between 4 and 6
(f) water to 1 g.

A preferred pharmaceutical formulation consists of:
(a) 0.29 μg to 234 μg active salt
(b) 0.21 to 21 mg citric acid
(c) 8 to 10 mg NaCl
(d) NaOH to adjust pH to between 5 and 5.5
(f) water to 1 g.

The present invention also provides a process for preparing an aqueous aerosol formulation. According to the present process, the active salt, preferably the crystalline diHCl salt of the present invention, is dissolved in an acidic aqueous solution of the buffering agent, which is provided as the acid. The pH is then adjusted by addition of a base, such as NaOH. For example, citric acid is added to a 0.9% sodium chloride solution. The active agent is dissolved in the acidic saline solution; providing a solution with an initial pH of around 2.5. The pH is then adjusted by the gradual addition of 1N NaOH until the solution has the desired pH. A remaining amount of 0.9% sodium chloride solution is added to provide a desired total solution weight. When a surfactant is included in the formulation, the surfactant can be mixed with the sodium chloride solution prior to the introduction of the buffering agent.

Surprisingly, a nebulizer solution having 0.1 mg of active compound 1, per gram of solution, prepared as described above, has been demonstrated to be stable upon storage. In contrast to the situation reported in U.S. Pat. No. 6,040,344 with respect to formoterol tartrate, in the present case, no unacceptable chemical degradation was observed after four months of storage at room temperature, as determined by an assay method based on high presure liquid chromatography (HPLC). In addition, the free base composition of a nebulizer solution was essentially unchanged after storage for nine months at 5° C.

Synthesis of Active Agent

The active agent, N-{2-[4-((R)-2-hydroxy-2-phenylethylamino)phenyl]ethyl}-(R)-2-hydroxy-2-(3-formamido-4-hydroxyphenyl)ethylamine, compound 1, can be synthesized from readily available starting materials as shown in the following Scheme and further described in the Examples below. It will be appreciated that while specific process conditions (i.e. reaction temperatures, times, mole ratios of reactants, solvents, pressures, etc.) are given, other process conditions can also be used unless otherwise stated.

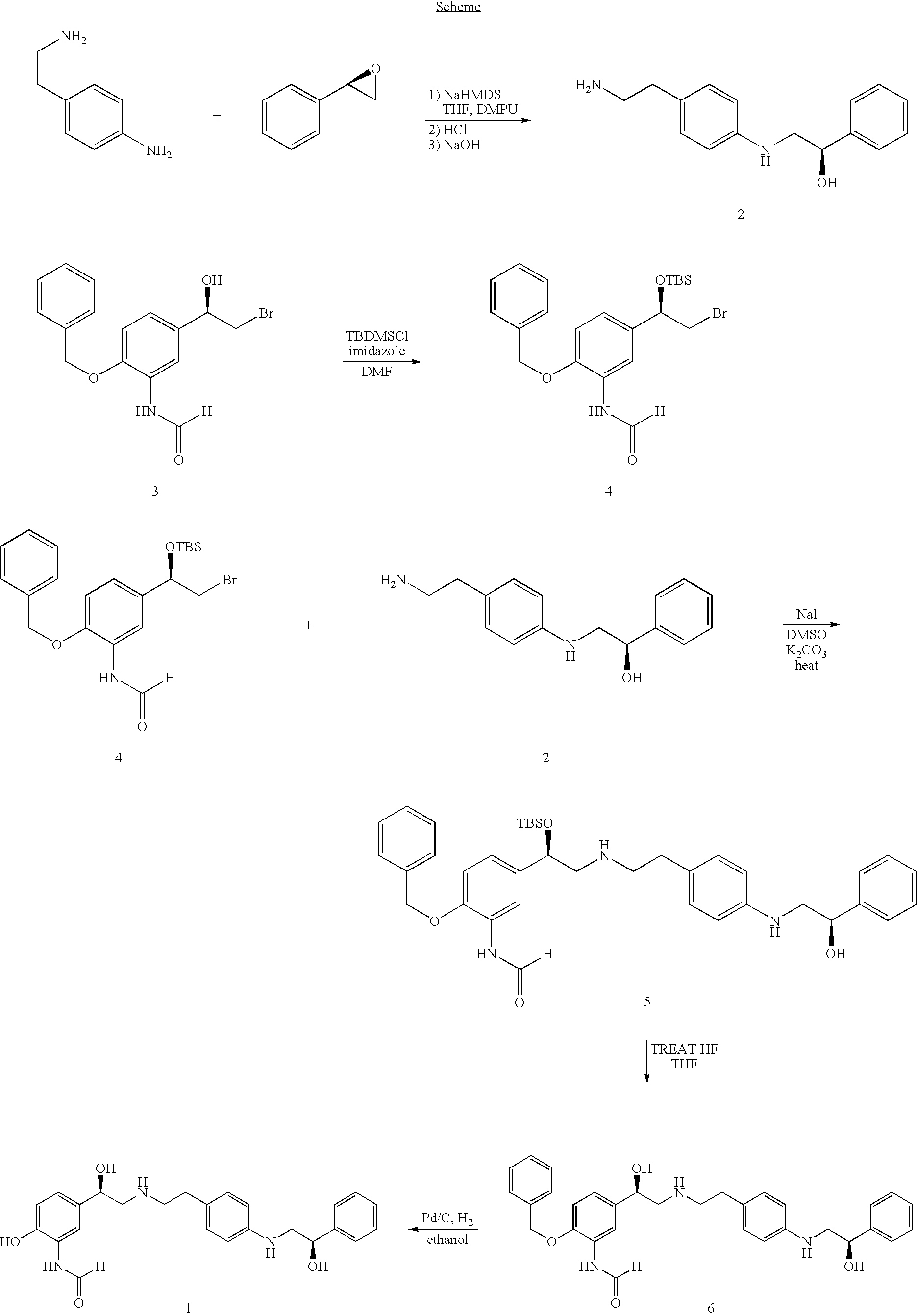

Intermediate 2 can be prepared by the coupling of 2-(4-aminophenyl)ethylamine and (R)-styrene oxide. The amine, which is optionally provided as a salt, is first reacted with between about 1 and about 1.2 equivalents of a base having a pKₐ value greater than about 18, in order to substantially deprotonate the 4-amino group. The (R)-styrene oxide is added to the product of the amine reaction. It has been observed that reactant stoichiometry can affect the purity of the product of this reaction. To ensure complete consumption of the (R)-styrene oxide, preferably less than one equivalent of the styrene oxide reactant is used. For example, a stoichiometry of 1.0 equivalent of 2-(4 -aminophenyl)ethylamine, 1.15 equivalent of base, and 0.95 equivalents of (R)-styrene oxide was found to be effective.

Useful basic compounds include sodium bis(trimethylsilyl)amide, alternatively known as sodium hexamethyldisilazane (NaHMDS), lithium diisopropyl amide, and n-butyl lithium. The reaction is preferably conducted in a solvent system including a polar aprotic solvent, such as 1,3-dimethyl-3,4,5,6-tetrahydro-2(1H)pyrimidinone (DMPU). Additional examples of aprotic polar solvents include dimethylsulfoxide, N-methylpyrollidinone, N,N-dimethyl acetamide, tetramethylethylenediamine, and hexamethylphosphoramide.

In the process according to the present invention, no protecting groups are required on the reactants. In addition, under the present conditions, the desired regioisomer is formed in significant quantities. When a strong base capable of deprotonating the aniline is not included, the reaction gives, primarily, the undesired regioisomer resulting from endo opening of the epoxide. Inclusion of the polar aprotic solvent in the solvent system allows the anion formed from deprotonation of the aniline to remain in solution.

After aqueous extraction, the product of the coupling reaction is crystallized as the hydrochloride salt from a solvent such as isopropanol, by the addition of aqueous hydrochloric acid. The crystallization procedure efficiently separates the desired product from side products formed during the reaction. The hydrochloride salt is redissolved with 10 N aqueous sodium hydroxide to provide 2-[4-((R)-2-hydroxy-2-phenylethylamino)phenyl]ethylamine (2).

The corresponding (S) stereoisomer, 2-[4-((S)-2-hydroxy-2-phenylethylamino)phenyl]ethylamine, can be prepared by substituting (S)-styrene oxide for (R)-styrene oxide in the above procedure for the synthesis of intermediate 2.

(R)-2-Bromo-1-(3-formamido-4-benzyloxyphenyl)ethanol (3), can be prepared as described in U.S. Pat. No. 6,268,533 B1; and in R. Hett et al., Organic Process Research and Development, 1998, 2, 96-99. Intermediate 3 can also be prepared using procedures similar to those described by Hong et al., Tetrahedron Lett., 1994, 35, 6631; or similar to those described in U.S. Pat. No. 5,495,054. Intermediate 4,2-bromo-(R)-1-tert-butyldimethylsiloxy-1-(3-formamido-4-benzyloxyphenyl)ethane, including the protecting group tert-butydimethylsilyl (TBS) at the hydroxyl position of 3, can be formed by the addition of tert-butydimethylsilylchloride (TBDMSCl) and imidazole to intermediate 3, dissolved in dimethylformamide (DMF).

Intermediates 2 and 4 are coupled, using dimethylsulfoxide (DMSO) as the solvent, by adding potassium carbonate and sodium iodide and heating to about 140° C. to form intermediate 5, N-{2-[4-((R)-2-hydroxy-2-phenylethylamino)phenyl]ethyl}-(R)-2-tert-butyldimethylsiloxy-2-(3-formamido-4-benzyloxyphenyl)ethylamine. The TBS protecting group is removed from 5, dissolved in tetrahydrofuran (THF), by addition of triethylamine trihydrofluoride (TREAT HF), giving intermediate 6, N-{2-[4-((R)-2-hydroxy-2-phenylethylamino)phenyl]ethyl}-(R)-2-hydroxy-2-(3-formamido-4-benzyloxyphenyl)ethylamine, upon isolation. The benzyl protecting group is removed from intermediate 6 by catalytic hydrogenolysis, using palladium on activated carbon, providing the active compound 1.

Pharmaceutical Compositions and Delivery Devices

The crystalline form of the diHCl salt of the present invention is advantageously used to prepare pharmaceutical compositions formulated for administration by inhalation. Pharmaceutical inhalation devices are generally classified as nebulizer inhalers, dry powder inhalers (DPI), and metered-dose inhalers (MDI). Conventional nebulizer devices produce a stream of high velocity air that causes a therapeutic agent to spray as a mist which is carried into the patient's respiratory tract. As disclosed above, an aqueous pharmaceutical composition formulated for nebulizer administration constitutes one aspect of the present invention. Alternatively, the therapeutic agent can be formulated for nebulizer administration as a suspension of micronized particles of respirable size, where micronized is typically defined as having about 90% or more of the particles with a diameter of less than about 10 μm.

Suitable nebulizer devices are provided commercially, for example, by PARI GmbH (Starnberg, Germany). Other nebulizer devices have been disclosed, for example, in U.S. Pat. No. 6,123,068. Aliquots of the aqueous formulations of the present invention are filled into sterile containers, for example, unit dose containers, suitable for delivery by the nebulizer devices. Thus, the present invention further provides a kit comprising a nebulizer device and a container whose contents comprise the present formulation. Depending on the actual delivery volume of such devices, the concentration of the formulation can be adjusted to provide an appropriate dose to the patient. Inclusion of a surfactant in the formulation can be beneficial in decreasing adsorption of the active agent on the nebulizer container, should such adsorption occur. Also the presence of the surfactant in the formulation can improve aerosolization in certain devices.

DPI's typically administer a therapeutic agent in the form of a free flowing powder that can be dispersed in a patient's air-stream during inspiration. Alternative DPI devices which use an external energy source to disperse the powder are also being developed. In order to achieve a free flowing powder, the therapeutic agent can be formulated with a suitable excipient (e.g., lactose or starch). A dry powder formulation can be made, for example, by combining dry lactose particles with micronized particles of the diHCl salt of compound 1 and dry blending. Alternatively, the agent can be formulated without excipients. The formulation is loaded into a dry powder dispenser, or into inhalation cartridges or capsules for use with a dry powder delivery device.

Examples of DPI delivery devices provided commercially include Diskhaler (GlaxoSmithKline, Research Triangle Park, N.C.) (see, e.g., U.S. Pat. No. 5,035,237); Diskus (GlaxoSmithKline) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI's typically discharge a measured amount of therapeutic agent using compressed propellant gas. Formulations for MDI administration include a solution or suspension of active ingredient in a liquefied propellant. While chlorofluorocarbons, such as CCl₃F, conventionally have been used as propellants, due to concerns regarding adverse affects of such agents on the ozone layer, formulations using hydrofluoroalklanes (HFA), such as 1,1,1,2-tetrafluoroethane (HFA 134a) and 1,1,1,2,3,3,3-heptafluoro-n-propane, (HFA 227) have been developed. Additional components of HFA formulations for MDI administration include co-solvents, such as ethanol, pentane, or minor amounts of water; and surfactants, such as sorbitan trioleate, oleic acid, lecithin, and glycerin. (See, for example, U.S. Pat. No. 5,225,183, EP 0717987 A2, and WO 92/22286).

Thus, a suitable formulation for MDI administration can include from about 0.001% to about 2% by weight of the present crystalline form, from about 0% to about 20% by weight ethanol, and from about 0% to about 5% by weight surfactant, with the remainder being the HFA propellant. In one approach, to prepare the formulation, chilled or pressurized hydrofluoroalkane is added to a vial containing the present crystalline form, ethanol (if present) and the surfactant (if present). To prepare a suspension, the pharmaceutical salt is provided as micronized particles. The formulation is loaded into an aerosol canister, which forms a portion of an MDI device. Examples of MDI devices developed specifically for use with HFA propellants are provided in U.S. Pat. Nos. 6,006,745 and 6,143,227.

In an alternative preparation, a suspension formulation is prepared by spray drying a coating of surfactant on micronized particles of the present crystalline material. (See, for example, WO 99/53901 and WO 00/61108). For additional examples of processes of preparing respirable particles, and formulations and devices suitable for inhalation dosing see U.S. Pat. Nos. 6,268,533, 5,983,956, 5,874,063, and 6,221,398, and WO 99/55319 and WO 00/30614.

The present active agent, compound 1, is effective over a range of dosages and is generally administered in a therapeutically effective amount. It will be understood, however, that the amount of the compound actually administered will be determined by a physician, in the light of the relevant circumstances, including the condition to be treated, the chosen route of administration, the age, weight, and response of the individual patient, the severity of the patient's symptoms, and the like.

Suitable doses of the therapeutic agent for inhalation administration are in the general range of from about 0.05 μg/day to about 1000 μg/day, preferably from about 0.1 μg/day to about 500 μg/day. It will be understood that the fraction of active agent delivered to the lung characteristic of particular delivery devices is taken into account in determining suitable doses for inhalation administration.

A compound can be administered in a periodic dose: weekly, multiple times per week, daily, or multiple doses per day. The treatment regimen may require administration over extended periods of time, for example, for several weeks or months, or the treatment regimen may require chronic administration. Suitable doses for oral administration are in the general range of from about 0.05 μg/day to about 100 mg/day, preferably from about 0.5 μg/day to about 1000 μg/day.

The invention thus provides a method of treating a disease or condition in a mammal associated with β₂ adrenergic receptor activity comprising administering to the mammal a therapeutically effective amount of the crystalline dihydrochloride salt of compound 1 or of a pharmaceutical composition comprising the crystalline dihydrochloride salt of compound 1.

The present active agent can also be co-administered with one or more other therapeutic agents. For example, the present agent can be administered in combination with one or more therapeutic agents selected from anti-inflammatory agents (e.g. corticosteroids and non-steroidal anti-inflammatory agents (NSAIDs), antichlolinergic agents (particularly muscarinic receptor antagonists), other β₂ adrenergic receptor agonists, antiinfective agents (e.g. antibiotics or antivirals) or antihistamines. The invention thus provides, in a further aspect, a combination comprising the dihydrochloride salt of compound 1 together with one or more therapeutic agent, for example, an anti-inflammatory agent, an antichlolinergic agent, another β₂ adrenergic receptor agonist, an antiinfective agent or an antihistamine.

The other therapeutic agents can be used in the form of pharmaceutically acceptable salts or solvates. As appropriate, the other therapeutic agents can be used as optically pure stereoisomers.

Suitable anti-inflammatory agents include corticosteroids and NSAIDs. Suitable corticosteroids which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Suitable NSAIDs include sodium cromoglycate; nedocromil sodium; phosphodiesterase (PDE) inhibitors (e.g. theophylline, PDE4 inhibitors or mixed PDE3/PDE4 inhibitors); leukotriene antagonists (e.g. monteleukast); inhibitors of leukotriene synthesis; iNOS inhibitors; protease inhibitors, such as tryptase and elastase inhibitors; beta-2 integrin antagonists and adenosine receptor agonists or antagonists (e.g. adenosine 2a agonists); cytokine antagonists (e.g. chemokine antagonists such as, an interleukin antibody (αIL antibody), specifically, an αIL-4 therapy, an αIL-13 therapy, or a combination thereof); or inhibitors of cytokine synthesis. Suitable other β₂-adrenoreceptor agonists include salmeterol (e.g. as the xinafoate), salbutamol (e.g. as the sulphate or the free base), formoterol (e.g. as the fumarate), fenoterol or terbutaline and salts thereof.

Also of interest is use of the present active agent in combination with a phosphodiesterase 4 (PDE4) inhibitor or a mixed PDE3/PDE4 inhibitor. The PDE4-specific inhibitor useful in this aspect of the invention may be any compound that is known to inhibit the PDE4 enzyme or which is discovered to act as a PDE4 inhibitor, and which are only PDE4 inhibitors. Preferred compounds are cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol].

Other compounds of interest include:

Compounds set out in U.S. Pat. No. 5,552,438 issued Sep. 3, 1996; this patent and the compounds it discloses are incorporated herein by reference. The compound of particular interest, which is disclosed in U.S. Pat. No. 5,552,438, is cis-4-cyano-4-[3-(cyclopentyloxy)-4-methoxyphenyl]cyclohexane-1-carboxylic acid (also known as cilomalast) and its salts, esters, pro-drugs or physical forms;

AWD-12-281 from elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (-)-p-[(4aR*,10bS*)-9-ethoxy-1,2,3,4,4a,10b-hexahydro-8-methoxy-2-methylbenzo[[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vemalis; or T-440 (Tanabe Seiyaku; Fuji, K. et al. J Pharmacol Exp Ther, 1998, 284(1): 162), and T2585.

Other possible PDE-4 and mixed PDE3/PDE4 inhibitors include those listed in WO0/13953, the disclosure of which is hereby incorporated by reference.

Suitable anticholinergic agents are those compounds that act as antagonists at the muscarinic receptor, in particular those compounds which are antagonists of the M₁, M₂, or M₃ receptors, or of combinations thereof. Exemplary compounds include the alkaloids of the belladonna plants as illustrated by the likes of atropine, scopolamine, homatropine, hyoscyamine; these compounds are normally administered as a salt, being tertiary amines. These drugs, particularly the salt forms, are readily available from a number of commercial sources or can be made or prepared from literature data via, to wit:

Atropine—CAS-51-55-8 or CAS-51-48-1 (anhydrous form), atropine sulfate—CAS-5908-99-6; atropine oxide—CAS-4438-22-6 or its HCl salt—CAS-4574-60-1 and methylatropine nitrate—CAS-52-88-0.

Homatropine—CAS-87-00-3, hydrobromide salt—CAS-51-56-9, methylbromide salt—CAS-80-49-9.

Hyoscyamine (d,l)—CAS-101-31-5, hydrobromide salt—CAS-306-03-6 and sulfate salt—CAS-6835-16-1.

Scopolamine—CAS-51-34-3, hydrobromide salt—CAS-6533-68-2, methylbromide salt—CAS-155-41-9.

Preferred anticholinergics include ipratropium (e.g. as the bromide), sold under the name Atrovent, oxitropium (e.g. as the bromide) and tiotropium (e.g. as the bromide) (CAS-139404-48-1). Also of interest are: methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO01/04118, the disclosure of which is hereby incorporated by reference.

Suitable antihistamines (also referred to as H₁-receptor antagonists) include any one or more of the numerous antagonists known which inhibit H¹-receptors, and are safe for human use. All are reversible, competitive inhibitors of the interaction of histamine with H₁-receptors. The majority of these inhibitors, mostly first generation antagonists, are characterized, based on their core structures, as ethanolamines, ethylenediamines, and alkylamines. In addition, other first generation antihistamines include those which can be characterized as based on piperizine and phenothiazines. Second generation antagonists, which are non-sedating, have a similar structure-activity relationship in that they retain the core ethylene group (the alkylamines) or mimic a tertiary amine group with piperizine or piperidine. Exemplary antagonists are as follows:

Ethanolamines: carbinoxamine maleate, clemastine fumarate, diphenylhydramine hydrochloride, and dimenhydrinate.

Ethylenediamines: pyrilamine amleate, tripelennamine HCl, and tripelennamine citrate.

Alkylamines: chlorpheniramine and its salts such as the maleate salt, and acrivastine.

Piperidines: hydroxyzine HCl, hydroxyzine pamoate, cyclizine HCl, cyclizine lactate, meclizine HCl, and cetirizine HCl.

Piperidines: Astemizole, levocabastine HCl, loratadine or its descarboethoxy analogue, and terfenadine and fexofenadine hydrochloride or another pharmaceutically acceptable salt.

Azelastine hydrochloride is yet another H₁ receptor antagonist which may be used in combination with the present active agent.

Examples of preferred anti-histamines include methapyrilene and loratadine.

The invention thus provides, in a further aspect, a combination comprising the crystalline dihydrochloride salt of compound 1 and a corticosteroid.

In particular, the invention provides combinations comprising the crystalline dihydrochloride salt of compound 1 and fluticasone propionate; the crystalline dihydrochloride salt of compound 1 and 6α,9α-difluoro-17α-[(2-furanylcarbonyl)oxy]-11β-hydroxy-16α-methyl-3-oxo-androsta-1,4-diene-17β-carbothioic acid S-fluoromethyl ester; and the crystalline dihydrochloride salt of compound 1 and 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester.

The invention thus provides, in a further aspect, a combination comprising the crystalline dihydrochloride salt of compound 1 and a PDE4 inhibitor.

The invention thus provides, in a further aspect, a combination comprising the crystalline dihydrochloride salt of compound 1 and an anticholinergic agent.

The invention thus provides, in a further aspect, a combination comprising the crystalline dihydrochloride salt of compound 1 and an antihistamine.

The invention thus provides, in a further aspect, a combination comprising the crystalline dihydrochloride salt of compound 1 together with a PDE4 inhibitor and a corticosteroid.

The invention thus provides, in a further aspect, a combination comprising the crystalline dihydrochloride salt of compound 1 together with an anticholinergic agent and a corticosteroid.

Accordingly, the pharmaceutical compositions of the invention can optionally comprise combinations of the crystalline dihydrochloride salt of compound 1 with one or more other therapeutic agents, as described above.

The other therapeutic agents can be provided in the form of aqueous solutions or suspensions. For example, a nebulizable suspension formulation of fluticasone propionate is described in U.S. Pat. No. 5,993,781. Accordingly, the aqueous aerosol pharmaceutical compositions of the invention can optionally comprise another therapeutic agent in solution or suspension form, in addition to the dihydrochloride salt of compound 1.

The individual compounds of the combinations of the invention may be administered either sequentially or simultaneously in separate or combined pharmaceutical formulations. Appropriate doses of known therapeutic agents will be readily appreciated by those skilled in the art.

According to a further aspect, the invention provides a method of treating a disease or condition associated with β₂ adrenergic receptor activity in a mammal, comprising administering to the mammal a therapeutically effective amount of a combination of the crystalline dihydrochloride salt of compound 1 with one or more other therapeutic agents.

Further, the present crystalline salt, potentially can be formulated for other forms of administration, such as oral or parenteral administration. The salt can be admixed with conventional pharmaceutical carriers and excipients and used in the form of powders, tablets, capsules, elixirs, suspensions, syrups, wafers, and the like. Such pharmaceutical compositions will contain from about 0.05 to about 90% by weight of the active compound, and more generally from about 0.1 to about 30%. Additional suitable pharmaceutical carriers for formulation of the crystalline salt of the present invention can be found in Remington: The Science and Practice of pharmacy, 20th Edition, Lippincott Williams & Wilkins, Philadelphia, Pa., 2000.

The following non-limiting examples illustrate representative pharmaceutical compositions of the invention, where active ingredient is defined as crystalline N-{2-[4-((R)-2-hydroxy-2-phenylethylamino)phenyl]ethyl}-(R)-2-hydroxy-2-(3-formamido-4-hydroxyphenyl)ethylamine dihydrochloride.

### FORMULATION EXAMPLE 1

An aqueous aerosol formulation for nebulizer administration having 0.1 mg of the active compound, compound 1, per gram of solution was prepared by the following procedure. Citric acid (755.8 mg) was added to a vessel charged with 0.9% sodium chloride solution (325.7 g). The active ingredient (42.3 mg) was added to the sodium chloride solution and the resulting mixture was stirred for 5 min and sonicated for 7 min to dissolve the active ingredient. The intial pH of the solution was determined to be 2.54. 1 N NaOH (7.1 g) was slowly added to the solution to obtain a final pH of 5.00. An additional amount of 0.9% sodium chloride solution (26.7 g) was added and the resulting solution was stirred to provide the aqueous formulation (360.3 g).

### FORMULATION EXAMPLE 2

To prepare 1 g of an aqueous aerosol formulation for nebulizer administration having 0.15 mg of the active compound, compound 1, per gram of solution, citric acid (2.1 mg) is added to a vessel charged with 0.9% sodium chloride solution (0.9 g). The active ingredient (0.1755 mg) is added to the sodium chloride solution and the resulting mixture is stirred and sonicated until the active ingredient is dissolved. The intial pH of the solution is about 2.5. The pH of the solution is adjusted to 5.0 by the slow addition of 1N NaOH (19.6 mg). An additional amount of 0.9% sodium chloride solution (78.1 mg) is added to adjust the weight of the solution to 1 g, and the resulting solution is stirred.

### FORMULATION EXAMPLE 3

An aqueous aerosol formulation with a concentration of 10 μg of compound 1 per gram of solution can be formulated following the procedure of Example 2 using the following components:

| | |
|---|---|
| Active ingredient | 0.0117 mg |
| Citric acid | 2.10 mg |
| 1N NaOH | q.s. to pH 5.0 |
| 0.9% NaCl solution | q.s. to 1 gram |

### FORMULATION EXAMPLE 4

An aqueous aerosol formulation with a concentration of 0.15 mg of compound 1 per gram of solution can be formulated following the procedure of Example 2 using the following components:

| | |
|---|---|
| Active ingredient | 0.1755 mg |
| Phosphoric acid | 1.07 mg |
| 1N NaOH | q.s. to pH 5.0 |
| 0.9% NaCl solution | q.s. to 1 gram |

### FORMULATION EXAMPLE 5

To prepare 1 g of an aqueous aerosol formulation for nebulizer administration having 0.15 mg of the active compound, compound 1, per gram of solution, Tween® 80 (0.01 mg) is mixed with 0.9% sodium chloride solution (0.9 g) in a vessel. Citric acid (2.1 mg) is added to the sodium chloride solution, and, then, the active ingredient (0.1755 mg) is added. The resulting mixture is stirred and sonicated until the active ingredient is dissolved. The intial pH of the solution is about 2.5. The pH of the solution is adjusted to 5.0 by the slow addition of 1N NaOH (19.6 mg). An additional amount of 0.9% sodium chloride solution is added to adjust the weight of the solution to 1 g, and the resulting solution is stirred.

### FORMULATION EXAMPLE 6

An aqueous aerosol formulation with a concentration of 0.15 mg of compound 1 per gram of solution can be formulated following the procedure of Example 5 using the following components:

| | |
|---|---|
| Active ingredient | 0.1755 mg |
| Citric acid | 2.10 mg |
| Tween ® 80 | 0.05 mg |
| 1N NaOH | q.s. to pH 5.0 |
| 0.9% NaCl solution | q.s. to 1 gram |

### FORMULATION EXAMPLE 7

### Stability Testing

Aliquots of the formulation prepared as in Example 1 were stored at −20° C., 5° C., room temperature, and 40° C. Samples were removed for analysis by HPLC at 1 month intervals. HPLC analysis was conducted using a Zorbax RP-bonus, C14, 25 cm×4.6 mm column, equilibrated at 35° C. The mobile phases used were: A: 0.1% TFA in 98:2 water:acetonitrile; and B: 0.1% TFA in 10:90 water:acetonitrile. Detection was by UV absorbance at 215 nm. A flow rate of 1.0 mL/min and a gradient of 0 to 60% B over 20 minutes was utilized. The percent of active compound 1 remaining after the indicated period, normalized to the sample stored at −20° C. is given in Table 1 below.

TABLE 1
Percent of active compound remaining upon 1-4 months storage

| Temperature | 1 month | 2 months | 3 months | 4 months |
|---|---|---|---|---|
| 5° C. | 101 | 100 | 100 | 99 |
| Room | 99 | 99 | 97 | 96 |
| 40° C. | 94 | 87 | 72 | 74 |

In a second stability test, aliquots were prepared and stored as above. Samples were removed for analysis by HPLC at various intervals. HPLC analysis was conducted using a MAC MOD Ace-5, C18, 25 cm×4.6 mm, 5 μm column, equilibrated at 30° C. The mobile phases were as described above. Detection was by UV absorbance at 244 nm. The initial condition was 6% phase B. A flow rate of 1.0 mL/min and a gradient of 6 to 30% B over 25 min, 30% to 60% B in 10 min and 60% to 100% B in 2 min were utilized. The retention time of compound 1 was approximately 20 min. The percent of active compound 1 remaining after the indicated period, normalized to the sample stored at −20° C. is given in Table 2 below.

TABLE 2
Percent of active compound remaining upon 4-9 months storage

| Temperature | 4 months | 5 months | 6 months | 9 months |
|---|---|---|---|---|
| 5° C. | 99 | 99 | 100 | 99 |
| Room | 97 | 95 | 94 | 90 |
| 40° C. | 74 | 69 | 63 | |

### FORMULATION EXAMPLE 8

### Other Pharmaceutical Compositions

### FORMULATION EXAMPLE 8A

This example illustrates the preparation of a representative pharmaceutical composition for oral administration of the crystalline diHCl salt of this invention:

| | Quantity per |
|---|---|
| Ingredients | capsule (mg) |
| Active Ingredient | 1 |
| Lactose, spray-dried | 148 |
| Magnesium stearate | 2 |

The above ingredients are mixed and introduced into a hard-shell gelatin capsule.

### FORMULATION EXAMPLE 8B

This example illustrates the preparation of another representative pharmaceutical composition for oral administration of the crystalline diHCl salt of this invention:

| | Quantity per |
|---|---|
| Ingredients | tablet, (mg) |
| Active Ingredient | 1 |
| Cornstarch | 50 |
| Lactose | 145 |
| Magnesium stearate | 5 |

The above ingredients are mixed intimately and pressed into single scored tablets.

### FORMULATION EXAMPLE 8C

This example illustrates the preparation of a representative pharmaceutical composition for oral administration of the crystalline diHCl salt of this invention.

An oral suspension is prepared having the following composition.

| Ingredients | | |
|---|---|---|
| Active Ingredient | 3 | mg |
| Fumaric acid | 0.5 | g |
| Sodium chloride | 2.0 | g |
| Methyl paraben | 0.1 | g |
| Granulated sugar | 25.5 | g |
| Sorbitol (70% solution) | 12.85 | g |
| Veegum K (Vanderbilt Co.) | 1.0 | g |
| Flavoring | 0.035 | mL |
| Colorings | 0.5 | mg |
| Distilled water | q.s. to 100 | mL |

### FORMULATION EXAMPLE 8D

This example illustrates the preparation of a representative pharmaceutical composition containing the crystalline diHCl salt of this invention.

An injectable preparation buffered to a pH of 4 is prepared having the following composition:

| Ingredients | |
|---|---|
| Active Ingredient | 0.1 mg |
| Sodium Acetate Buffer Solution (0.4 M) | 2.0 mL |
| HCl (1N) | q.s. to pH 4 |
| Water (distilled, sterile) | q.s. to 20 mL |

### FORMULATION EXAMPLE 8E

This example illustrates the preparation of a representative pharmaceutical composition for injection using the crystalline diHCl salt of this invention.

A reconstituted solution is prepared by adding 20 mL of sterile water to 1 mg of the compound of this invention. Before use, the solution is then diluted with 200 mL of an intravenous fluid that is compatible with the active compound. Such fluids are chosen from 5% dextrose solution, 0.9% sodium chloride, or a mixture of 5% dextrose and 0.9% sodium chloride. Other examples are lactated Ringer's injection, lactated Ringer's plus 5% dextrose injection, Normosol-M and 5% dextrose, Isolyte E, and acylated Ringer's injection.

### FORMULATION EXAMPLE 8F

This example illustrates the preparation of a representative pharmaceutical composition for topical application of the crystalline diHCl salt of this invention.

| Ingredients | grams |
|---|---|
| Active ingredient | 0.2-10 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

### FORMULATION EXAMPLE 8G

This example illustrates the preparation of a dry powder formulation containing a the diHCl salt of the invention for use in inhalation cartridges.

Inhalation cartridges are filled with a pharmaceutical composition having the following ingredients:

| Ingredients | mg/cartridge |
|---|---|
| Active ingredient | 0.2 |
| Lactose | 25 |

The active ingredient is micronized prior to blending with lactose. The contents of the cartridges are administered using a powder inhaler.

### FORMULATION EXAMPLE 8H

This example illustrates the preparation of a dry powder formulation containing the crystalline diHCl salt of this invention for use in a dry powder inhalation device.

A pharmaceutical composition is prepared having a bulk formulation ratio of micronized active ingredient to lactose of 1:200. The composition is packed into a dry powder inhalation device capable of delivering 25 μg of active drug ingredient per dose.

### FORMULATION EXAMPLE 8I

This example illustrates the preparation of a formulation containing the crystalline diHCl salt of this invention for use in a metered dose inhaler.

A suspension containing 5% active ingredient, 0.5% lecithin, and 0.5% trehalose is prepared by dispersing 5 g of active compound as micronized particles with mean size less than 10 μm in a colloidal solution formed from 0.5 g of trehalose and 0.5 g of lecithin dissolved in 100 mL of demineralized water. The suspension is spray dried and the resulting material is micronized to particles having a mean diameter less than 1.5 μm. The particles are loaded into canisters with pressurized 1,1,1,2-tetrafluoroethane.

### FORMULATION EXAMPLE 8J

This example illustrates the preparation of a formulation containing the crystalline diHCl salt of this invention for use in a metered dose inhaler.

A suspension containing 5% active ingredient and 0.1% lecithin is prepared by dispersing 10 g of active compound as micronized particles with mean size less than 10 μm in a solution formed from 0.2 g of lecithin dissolved in 200 mL of demineralized water. The suspension is spray dried and the resulting material is micronized to particles having a mean diameter less than 1.5 μm. The particles are loaded into canisters with pressurized 1,1,1,2,3,3,3-heptafluoro-n-propane.

### FORMULATION EXAMPLES 8K-80

Formulation Examples 8K-80 illustrate suspension aerosol formulations containing suspensions of micronized particles of the diHCl salt of the present invention in a hydrofluoroalkane propellant for use in a metered dose inhaler. To prepare the formulation, chilled or pressurized hydrofluoroalkane is added to a vial containing the other ingredients. The formulation is loaded into aerosol canisters.

% by weight

Formulation Example 8K

| | |
|---|---|
| Active ingredient | 0.4 |
| Oleic acid | 0.5 |
| Ethanol | 15 |
| 1,1,1,2-tetrafluoroethane | remainder |

Formulation Example 8L

| | |
|---|---|
| Active ingredient | 0.4 |
| Sorbitan trioleate | 0.1 |
| Ethanol | 10 |
| 1,1,1,2,3,3,3-heptafluoro-n-propane | remainder |

Formulation Example 8M

| | |
|---|---|
| Active ingredient | 0.4 |
| Oleic acid | 0.5 |
| Ethanol | 15 |
| 1,1,1,2,3,3,3-heptafluoro-n-propane | remainder |

Formulation Example 8N

| | |
|---|---|
| Active ingredient | 0.4 |
| 1,1,1,2,3,3,3-heptafluoro-n-propane | remainder |

Formulation Example 8O

| | |
|---|---|
| Active ingredient | 0.4 |
| Lecithin | 0.5 |
| Ethanol | 15 |
| 1,1,1,2,3,3,3-heptafluoro-n-propane | remainder |

The following exemplify the preparation of N-{2-[4-((R)-2-hydroxy-2-phenylethylamino)phenyl]ethyl}-(R)-2-hydroxy-2-(3-formamido-4-hydroxyphenyl)ethylamine, compound 1; and preparation, characterization, and stability testing of the crystalline diHCl salt of compound 1.

### EXAMPLE 1a

### Synthesis of 2-[4-((R)-2-hydroxy-2-phenylethylamino)phenyl]ethylamine (2)

To a 1000 mL 3-neck flask was added 10 g (74 mmol) of 2-(4-aminophenyl)ethylamine and 15 mL of 1,3-dimethyl-3,4,5,6-tetrahydro-2(1H)pyrimdinone (DMPU). The reaction flask was fitted with an overhead stirrer, a 125 mL addition funnel and a thermometer. The reaction flask was purged with nitrogen and placed in a cold water bath. The addition funnel was charged with 83 mL (83 mmol) of 1.0 M sodium bis(trimethylsilyl)amide in tetrahydrofuran. The sodium bis(trimethylsilyl)amide solution was added dropwise over 30 min with vigorous stirring. The addition funnel was removed and replaced with a rubber septum. (R)-styrene oxide (8.4 mL, 74 mmol) was added dropwise by syringe over 10 minutes. The rate of addition was controlled to maintain a temperature below 35° C. After 1 h, the reaction was quenched by dropwise addition of 88 mL water. The reaction mixture was transferred to a separatory funnel, diluted with 56 mL isopropyl acetate and washed with 84 mL saturated aqueous sodium chloride. The organic layer was washed a second time with a mixture of 84 mL water and 84 mL saturated aqueous sodium chloride and finally with 84 mL saturated aqueous sodium chloride. The organic layer was concentrated under vacuum. The residue was twice reconcentrated from isopropanol (55 mL portions) and then redissolved in isopropanol (235 mL) and heated to 70° C. with stirring. Concentrated hydrochloric acid (13.2 mL, 160 mmol) was added over two minutes. The mixture was allowed to cool to room temperature and stirred for 14 h. The precipitated product was isolated by filtration and washed with isopropanol and isopropyl acetate. The product was dried under vacuum for 3 h and then dissolved in 56 mL water and transferred to a separatory funnel. Isopropyl acetate (56 mL) and 10 N aqueous sodium hydroxide (19 mL, 190 mmol) were added. The separatory funnel was shaken and the phases separated. The organic layer was dried over sodium sulfate and concentrated to afford the product 2 as an orange-brown oil (11 g, 44 mmol, 59%). m/z: [M+H⁺] calcd for C₁₆H₂₀N₂O 257.2; found 257.2.

### EXAMPLE 1b

### Synthesis of 2-[4-((R)-2-hydroxy-2-phenylethylamino)phenyl]ethylamine (2) Dihydrochloride

To a 500 mL 3-neck flask was added 10 g (74 mmol) of 2-(4-aminophenyl)ethylamine and 15.2 mL of 1,3-dimethyl-3,4,5,6-tetrahydro-2(1H)pyrimdinone (DMPU). The reaction flask was fitted with an overhead stirrer, a 125 mL addition funnel and a thermometer. The reaction flask was purged with nitrogen and placed in a cold water bath. The addition funnel was charged with 84.4 mL (84.4 mmol, 1.15 equiv.) of 1.0 M sodium bis(trimethylsilyl)amide in tetrahydrofuran. The sodium bis(trimethylsilyl)amide solution was added dropwise over 30 min with vigorous stirring. The addition funnel was removed and replaced with a rubber septum. (R)-styrene oxide (8.0 mL, 70.3 mmol, 0.95 equiv.) was added dropwise by syringe over 15 minutes. The rate of addition was controlled to maintain a temperature below 35° C. After 1 h, the reaction was quenched by dropwise addition of 90 mL water. The reaction mixture was transferred to a separatory funnel, diluted with 60 mL isopropyl acetate and washed with 90 mL saturated aqueous sodium chloride. The organic layer was washed a second time with a mixture of 90 mL water and 90 mL saturated aqueous sodium chloride and finally with 90 mL saturated aqueous sodium chloride. The organic layer was concentrated under vacuum. The residue was concentrated twice from isopropanol (51 mL portions) and then redissolved in isopropanol (250 mL) and heated to 70° C. with stirring. Concentrated hydrochloric acid (13.2 mL, 160 mmol) was added over two minutes. The mixture was allowed to cool to room temperature and stirred for 12 h. The precipitated product was isolated by filtration, washed with isopropanol and isopropyl acetate, and dried under vacuum for three hours to yield 13.67 g (57% yield) of 2-[4-((R)-2-hydroxy-2-phenylethylamino)phenyl]ethylamine (2) dihydrochloride as a white solid.

### EXAMPLE 2

### Synthesis of 2-bromo-(R)-1-tert-butyldimethylsiloxy-1-(3-formamido-4-benzyloxyphenyl)ethane (4)

(R)-2-Bromo-1-(3-formamido-4-benzyloxyphenyl)ethanol (intermediate 3)(9.9 g, 28 mmol) was dissolved in 36 mL dimethylformamide. Imidazole (2.3 g, 34 mmol) and t-butyldimethylsilylchloride (4.7 g, 31 mmol) were added. The solution was stirred under nitrogen atmosphere for 72 h. Additional imidazole (0.39 g, 5.7 mmol) and t-butyldimethylsilylchloride (0.64 g, 4.3 mmol) were added and the reaction was stirred for an additional 20 h. The reaction was diluted with a mixture of isopropyl acetate (53 mL) and hexanes (27 mL) and transferred to a separatory funnel. The organic layer was twice washed with a mixture of water (27 mL) and saturated aqueous sodium chloride (27 mL) followed by a final wash with saturated aqueous sodium chloride (27 mL). The organic layer was dried over sodium sulfate. Silica gel (23.6 g) and hexanes (27 mL) were added and the suspension was stirred for 10 minutes. The solids were removed by filtration and the filtrate concentrated under vacuum. The residue was crystallized from hexanes (45 mL) to afford 8.85 g (19 mmol, 68%) of intermediate 4 as a white solid. m/z: [M+H⁺] calcd for C₂₂H₃₀NO₃SiBr 464.1, 466.1; found 464.2, 466.4.

### EXAMPLE 3

### Synthesis of N-{2-[4-((R)-2-hydroxy-2-phenylethylamino)phenyl]ethyl}-(R)-2-tert-butyldimethylsiloxy-2-(3-formamido-4-benzyloxyphenyl)ethylamine (5)

Intermediate 4 (5.0 g, 11 mmol), intermediate 2 (3.5 g, 14 mmol), and dimethylsulfoxide (10 mL) were combined in a 100 mL round bottom flask and stirred to form a homogeneous solution. Potassium carbonate (6.0 g, 43 mmol) and sodium iodide (1.7 g, 11 mmol) were added and the reaction mixture was heated to 140° C. The reaction mixture was maintained at 140° C. for 10 min, then cooled to room temperature and diluted with water (24 mL) and isopropyl acetate (28 mL). The reaction was stirred until all solids dissolved and then transferred to a separatory funnel. The organic layer was washed with water (17 mL) followed by acetate buffer (5% v/v acetic acid, 12% w/v sodium acetate trihydrate in water, 18 ml) followed by sodium bicarbonate solution (5% w/v in water, 17 mL) followed by saturated aqueous sodium chloride (17 mL). The organic layer was dried over sodium sulfate and concentrated to afford intermediate 5 as a brown gelatinous solid (7.0 g, 11 mmol, >99%). m/z: [M+H⁺] calcd for C₃₈H₄₉N₃O₄Si 640.4; found 640.6.

### EXAMPLE 4

### Synthesis of N-{2-[4-((R)-2-hydroxy-2-phenylethylamino)phenyl]ethyl}-(R)-2-hydroxy-2-(3-formamido-4-benzyloxyphenyl)ethylamine (6)

Intermediate 5 (5.2 g, 8.1 mmol) was dissolved in tetrahydrofuran (26 mL) and triethylamine trihydrofluoride (1.4 mL, 8.6 mmol) was added. The solution was stirred for 20 h. The reaction was quenched by addition of water (7.6 mL) followed by 10.0 N sodium hydroxide (3.8 mL, 38 mmol). After 3 min, the reaction was diluted with isopropyl acetate (20 mL) and transferred to a separatory funnel. The mixture was shaken and the biphasic mixture was filtered through celite to remove undissolved solids. The filtrate was returned to a separatory funnel and the phases were separated. The organic layer was washed with a mixture of 9 mL water and 9 mL saturated aqueous sodium chloride followed by 15 mL of saturated aqueous sodium chloride. The organic layer was dried over sodium sulfate and concentrated to afford the product 6 as a brown gelatinous solid (4.2 g, 8.0 mmol, 99%). m/z: [M+H⁺] calcd for C₃₂H₃₅N₃O₄ 526.3; found 526.4.

### EXAMPLE 5

### Synthesis of N-{2-[4-((R)-2-hydroxy-2-phenylethylamino)phenyl]ethyl}-(R)-2-hydroxy-2-(3-formamido-4-hydroxyphenyl)ethylamine (1)

Intermediate 6 (2.5 g, 4.8 mmol) was dissolved in 8.0 mL of ethanol and treated with activated charcoal, Darco G-60 (1.25 g). The suspension was stirred at 50° C. for 20 min and then filtered to remove the Darco. To the filtrate was added 10% palladium on activated carbon (250 mg) and the suspension placed on a Parr shaker. The reaction was shaken for 10 h under 30 psi hydrogen gas. The reaction was filtered through celite and concentrated under vacuum to afford compound 1 as a brown gelatinous solid (1.9 g, 4.3 mmol, 91%). ¹H NMR (300 MHz, DMSO-d₆) δ 2.40-2.68 (m, 6H), 2.92-3.18 (m, 2H), 4.35-4.45 (m, 1H), 4.60-4.69 (m, 1H), 5.22-5.30 (m, 1H), 6.82 (s, 1H), 6.85 (s, 1H), 6.68-6.86 (m, 4H), 7.12-7.36 (m, 5H), 7.95 (d, 1H, J=1.4 Hz), 8.19 (s, 1H), 9.49 (br s, 1H). m/z: [M+H⁺] calcd for C₂₅H₂₉N₃O₄ 436.2; found 436.4.

### EXAMPLE 6

### Crystallization of N-{2-[4-((R)-2-hydroxy-2-phenylethylamino)phenyl] ethyl}-(R)-2-hydroxy-2-(3-formamido-4-hydroxyphenyl)ethylamine Dihydrochloride

Compound 1 (3.17 g, 7.3 mmol) was dissolved in 111 mL isopropanol in a bath at 50° C. and allowed to cool. The solution was stirred rapidly and 1.0 N HCl was added (24 mL, 24 mmol). The mixture was stirred for 6 h at room temperature. The colorless crystalline product was isolated by filtration and dried under vacuum to afford the dihydrochloride salt of compound 1 (1.71 g, 3.4 mmol, 46%).

### EXAMPLE 7a

### Recrystallization of N-{2-[4-((R)-2-hydroxy-2-phenylethylamino)phenyl]ethyl}-(R)-2-hydroxy-2-(3-formamido-4-hydroxyphenyl)ethylamine Dihydrochloride

The crystallized dihydrochloride salt of compound 1 from Example 6 (1.5 g, 3.0 mmol) was dissolved in 24 mL of 50% v/v aqueous isopropanol at 50° C. The warm solution was diluted with 48 mL isopropanol and stirred for 2 h. The recrystallized product was isolated by filtration and dried under vacuum to afford 1.0 g the dihydrochloride salt of compound 1 (2.0 mmol, 66%).

### EXAMPLE 7b

### Recrystallization of N-{2-[4-((R)-2-hydroxy-2-phenylethylamino)phenyl]ethyl}-(R)-2-hydroxy-2-(3-formamido-4-hydroxyphenyl)ethylamine Dihydrochloride

A 250 mL three-necked flask was charged with a hydrochloride salt of compound 1 having 1.52 equivalents of chlorine per mole of compound 1 (6.1 g, 12 mmol) and 1:1 isopropanol:water (98 mL). The mixture was heated to 50-60° C. to dissolve the solid. Hydrochloric acid (38%, 1.2 mL, 12 mmol) was added at 50-55° C. and then isopropanol (98 mL) was slowly added over 30 min at 50-55° C. The solution was cooled to room temperature over 2 h. Isopropanol (98 mL) was slowly added over 30 min and the solution was stirred at room temperature for 16 h. The crystals were isolated by filtration, washed with isopropanol (30 mL) and dried under vacuum for 24 h to yield the dihydrochloride salt of compound 1 (4.8 g, 9.4 mmol, 79% yield, 99.3% purity by HPLC)

The hydrochloride salt of compound 1 having 1.52 equivalents of chlorine per mole of compound 1 was obtained as follows. Intermediate 6 (3.1 kg), prepared according to the procedures of Examples 1 to 4 was debenzylated according to the process of Example 5, and then crystallized according to the process of Example 6a to afford 2.2 kg of a hydrochloride salt of compound 1. The crystallized product was recrystallized according to the process of Example 7a to afford 1.2 kg of a hydrochloride salt of compound 1 having 1.52 equivalents of chlorine.

### EXAMPLE 8

### Characterization of N-{2-[4-((R)-2-hydroxy-2-phenylethylamino)phenyl]ethyl}-(R)-2-hydroxy-2-(3-formamido-4-hydroxyphenyl)ethylamine Dihydrochloride

The x-ray powder diffraction pattern and differential scanning calorimetry trace of the crystalline diHCl salt of compound 1 crystallized from the free base as in Example 6, are shown in FIGS. 1 and 2, respectively. The sample of crystalline material which was analyzed to provide the results of FIGS. 1 and 2 had a characteristic particle size of 32.3±19.1 μm.

The characteristic IR peak positions were determined as the average position of the common peaks of three different samples of the diHCl salt: 696±1, 752±1, 787±1, 827±1, 873±1, 970±1, 986+1, 1020±1, 1055±1, 1066±1, 1101±1, 1197±1, 1293±1, 1371±1, 1440±1, 1542±1, 1597±1, 1658±1, 2952±1, 3372±1, and 3555±1cm⁻¹.

The diHCl salt of compound b 1 produced by the process of Examples 1 through 7a was characterized as follows:

IR: 697, 753, 787, 827, 873, 970, 986, 1021, 1055, 1065, 1100, 1198, 1294, 1371, 1440, 1541, 1597, 1658, 2499, 2771, 2953, 3371, 3555 cm⁻¹. ¹H NMR(300 MHz, DMSO-d₆) δ 2.86-3.37 (m, 8H), 4.80-4.91 (m, 2H), 6.91 (s, 2H), 7.12-7.43 (m, 9H), 8.13 (s, 1H), 8.27 (d, 1H, J=1.6 Hz), 8.80 (br s, 1H), 9.27 (br s, 1H), 9.63 (s, 1H), 10.18 (br s, 1H). ¹³C NMR (100 MHz, DMSO-d₆) δ 30.8, 47.8, 53.7, 56.3, 68.1, 68.4, 114.9, 118.4, 121.6, 121.9, 125.9, 126.0, 127.6, 128.2, 129.7, 132.2, 142.0, 146.4, 160.0. m/z: [M+H⁺] calcd for C₂₅H₂₉N₃O₄ 436.2; found 436.2. Elemental analysis (wt %) calcd for C₂₅H₂₉N₃O_{40.2}HCl: C, 59.06; H, 6.15; N, 8.26; O, 12.59; Cl, 13.95. found: C, 59.62; H, 6.28; N, 8.41; O, 11.71; Cl, 13.52.

### EXAMPLE 9

### Solid state stability testing of N-{2-[4-((R)-2-hydroxy-2-phenylethylamino)phenyl]ethyl}-(R)-2-hydroxy-2-(3-formamido-4-hydroxyphenyl)ethylamine Dihydrochloride

A single lot of the crystalline diHCl salt of compound 1]prepared as in Example 6, was used to prepare four samples of approximately 30 mg each. Two samples were ground to fine particles with a mortar and pestle. Two samples, one ground and one that had not been processed, were stored in a closed chamber at 40° C. and 75% relative humidity for 30 days and two samples, one ground and one that had not been processed, were stored at room temperature in a desiccators for the same period. Samples were withdrawn for analysis by DSC and TGA at 11 days and by XRPD and HPLC at 30 days.

Comparison of the DSC traces for the four samples indicates there is no detectable effect on crystal stability after exposure to elevated temperature and humidity. TGA results show no appreciable quantity of water becomes associated with either the ground or the unground crystals, after exposure to high relative humidity. Similarly, the XRPD patterns show that the samples are not appreciably affected by exposure to heat and humidity and no appreciable chemical degradation was observed in the HPLC data.

In another test, samples of the recrystallized diHCl salt of Example 7a were stored at 40° C. and 75% relative humidity. After 6 months, there was no visible contamination in the appearance of the material, the water content remained negligible (0.12% water initially, 0.37% water at 6 mo), the chemical purity of the sample, as determined by HPLC analysis was essentially unchanged, and there was no change in the chiral purity of the sample.

Analytical Methods

X-ray powder diffraction patterns were obtained with a Shimadzu 6000 diffractometer using Cu Kα (40.0 kV, 35.0 mA) radiation. The analysis was performed with the goniometer running in continuous-scan mode of 2°/min with a step size of 0.02° over a range of 4 to 45°. Samples were prepared on glass specimen holders as a thin layer of powdered material.

Differential scanning calorimetry traces were obtained with a TA instruments model DSC2010. Samples were placed in sealed aluminum pans for analysis with an empty pan serving as the reference. Samples were equilibrated at 30° C. and heated at 5° C. per minute to a temperature of 300° C. The instrument was calibrated with an indium standard.

Thermogravimetric analysis was conducted using a TA instruments model Q50. Samples were weighed in aluminum pans and heated from 50° C. to 300° C. at a rate of 10° C./min. Water content was estimated by measuring the total weight loss between 50° and 120° C. (a range in which no significant decomposition was observed).

The IR spectrum was determined over the wave number (υ) range 4000 to 675 cm⁻¹ using an Avatar 360 FT-IR spectrometer equipped with a Nicolet omnis sample attenuated total reflection (ATR) sample holder.

¹H NMR spectra were acquired on a 300 MHz Varian Gemini 2000 spectrometer at ambient temperature. Samples were dissolved in DMSO-d6 and chemical shifts were reported on a TMS scale using residual DMSO protons (2.49 ppm) as reference. ¹³C NMR spectra were acquired on JEOL Eclipse⁺ 400 MHz spectrometer.

Unless otherwise indicated, HPLC analysis was conducted using a Zorbax RP-bonus, C14, 25 cm×4.6 mm column, equilibrated at 35° C. The mobile phases used were: A: 0.1% TFA in 98:2 water:acetonitrile; and B: 0.1% TFA in 10:90 water:acetonitrile. Detection was by UV absorbance at 215 nm. A flow rate of 1.0 mL/min and a gradient of 0 to 60% B over 20 minutes was utilized. The diHCl salt of compound 1 gave a retention time of 11.7 min.

Mass spectrometric identification was performed by an electrospray ionization method (ESMS) with a Perkin Elmer instrument (PE SCIEX API 150 EX).

Chiral purity was determined using a Beckman P/ACE MDQ capillary electrophoresis system. The analysis was performed at pH 2.5 using heptakis-(2,3,-diacetyl-6-sulfato)-β-cyclodextrin (HDAS-β-CD) as the chiral selector and using a 50 μm×31.2 cm fused silica capillary. Detection was by UV absorbance at 200 nm. The four stereoisomers migrate in the following order: SS, RS, SR, RR, where compound 1 is designated as RR.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. Additionally, all publications, patents, and patent documents cited hereinabove are incorporated by reference herein in full, as though individually incorporated by reference.

1. Crystalline N-{2-[4-((R)-2-hydroxy-2-phenylethylamino)phenyl]ethyl}-(R)-2-hydroxy-2-(3-formamido-4-hydroxyphenyl)ethylamine dihydrochloride.

2. The compound of claim 1 which is characterized by an x-ray powder diffraction pattern having two or more diffraction peaks at 2θ values selected from the group consisting of 15.61±0.2, 16.32±0.2, 19.50±0.2, 24.25±0.2, 24.92±0.2, 25.45±0.2, 28.67±0.2, and 31.16±0.2.

3. The compound of claim 1 wherein the x-ray powder diffraction pattern comprises diffraction peaks at 2θ values of 24.25±0.2, 24.92±0.2, and 25.45±0.2.

4. The compound of claim 1 which is characterized by an x-ray powder diffraction pattern in which the peak positions are substantially in accordance with the peak positions of the pattern shown in FIG. 1.

5. The compound of claim 1 having an infrared absorption spectrum with significant absorption bands at 696±1, 752±1, 787±1, 827±1% 873±1, 970±1, 986±1, 1020±1, 1055±1, 1066±1, 1101±1, 1197±1, 1293±1, 1371±1, 1440±1, 1542±1, 1597±1, 1658±1, 2952±1, 3372±1, and 3555±1 cm⁻¹.

6. The compound of claim 1 which is characterized by a differential scanning calorimetry trace which shows an onset of endothermic heat flow at about 200° C.

7. A hydrochloride salt of N-{2-[4-((R)-2-hydroxy-2-phenylethylamino)phenyl]ethyl}-(R)-2-hydroxy-2-(3-formamido-4-hydroxyphenyl)ethylamine having an x-ray powder diffraction pattern having two or more diffraction peaks at 2θ values selected from the group consisting of 15.61±0.2, 16.32±0.2, 19.50±0.2, 24.25±0.2, 24.92±0.2, 25.45±0.2, 28.67±0.2, and 31.16±0.2.

8. A pharmaceutical composition comprising a therapeutically effective amount of the compound of claim 1 and a pharmaceutically acceptable carrier.

9. The pharmaceutical composition of claim 8, wherein the composition comprises particles of crystalline N-{2-[4-((R)-2-hydroxy-2-phenylethylamino)phenyl]ethyl}-(R)-2-hydroxy-2-(3-formamido-4-hydroxyphenyl)ethylamine dihydrochloride having a size ranging from about 1 μm to about 10 μm.

10. The pharmaceutical composition of claim 8, wherein the composition further comprises a therapeutically effective amount of one of more other therapeutic agents.

11. The pharmaceutical composition of claim 8, wherein the composition is formulated for administration by inhalation.

12. A combination comprising the compound of claim 1 and one or more other therapeutic agents.

13. The combination of claim 12 wherein the other therapeutic agent is a corticosteroid, an antichlolinergic agent, or a PDE4 inhibitor.

14. A combination comprising a compound of Claim 1 and 6α,9α-difluoro-17α-[(2-furanylcarbonyl)oxy]-11α-hydroxy-16α-methyl-3-oxo-androsta-1,4-diene-17β-carbothioic acid S-fluoromethyl ester or 6α,9αdifluoro-11β-hydroxy-16α-methyl-3-oxo-17α-propionyloxy-androsta-1,4-diene-17β-carbothioic acid S-(2-oxo-tetrahydro-furan-3S-yl) ester.

15. A process for preparing crystalline N-{2-[4-((R)-2-hydroxy-2-phenylethylamino)phenyl]ethyl}-(R)-2-hydroxy-2-3-formamido-4-hydroxyphenyl)ethylamine dihydrochloride, the process comprising the steps of:
(a) dissolving N-{2-[4-((R)-2-hydroxy-2-phenylethylamino)phenyl]ethyl}-(R)-2-hydroxy-2-(3-formamido-4-hydroxyphenyl)ethylamine in a first polar solvent to form a first solution; and
(b) adding hydrochloric acid to form a second solution from which a crystalline product is formed.

16. The process of claim 15 wherein the second solution comprises isopropanol and water in a ratio of isopropanol:water of from about 4:1 to about 10:1, volume to volume.

17. The process of claim 15 further comprising:
(a) dissolving the product of claim 15 in a second polar solvent; and
(b) adding between about 0.5 and about 1.5 equivalents of hydrochloric acid per mole of free base and a third polar solvent to form a third solution from which a crystalline product is formed.

18. The crystalline hydrochloride salt produced by the process of claim 15.

19. The crystalline hydrochloride salt of claim 18 wherein the salt has an x-ray powder diffraction pattern having two or more diffraction peaks at 2θ values selected from the group consisting of 15.61±0.2, 16.32±0.2, 19.50±0.2, 24.25±0.2, 24.92±0.2, 25.45±0.2, 28.67±0.2, and 31.16±0.2.

20. A pharmaceutical composition comprising:
(a) N-{2-[4-((R)-2-hydroxy-2-phenylethylamino)phenyl]ethyl}-(R)-2-hydroxy-2-(3-formamido-4-hydroxyphenyl)ethylamine dihydrochloride;
(b) a buffering agent; and
(c) water;
wherein the buffering agent is present in an amount sufficient to provide the composition with a pH in the range of between about 4 and about 6.

21. The pharmaceutical composition of claim 20 wherein the buffering agent is present in an amount sufficient to provide the composition with a pH in the range of between about 5 and about 5.5.

22. The pharmaceutical composition of claim 20 where the buffering agent comprises a citrate species.

23. The pharmaceutical composition of claim 20 wherein the composition is isotonic.

24. The pharmaceutical composition of claim 23 wherein the composition further comprises a sufficient amount of sodium chloride to render the composition isotonic.

25. The pharmaceutical composition of claim 20, wherein the composition further comprises a surfactant.

26. The pharmaceutical composition of claim 20, wherein the composition further comprises a therapeutically effective amount of one or more other therapeutic agents.

27. A kit comprising:
(a) a nebulizer device; and
(b) a container whose contents comprise the pharmaceutical composition of claim 20.

28. A process for preparing a pharmaceutical composition for use in a nebulizer, the process comprising the steps of:
(a) dissolving crystalline N-{2-[4-((R)-2-hydroxy-2-phenylethylamino)phenyl]ethyl}-(R)-2-hydroxy-2-(3-formamido-4-hydroxyphenyl)ethylamine dihydrochloride in an acidic aqueous solution comprising a buffering agent; and
(b) adding a base until the composition has a pH of between about 4 and about 6.

29. The process of claim 28 wherein the acidic aqueous solution is an isotonic solution.

30. The process of claim 28 wherein step (b) comprises adding NaOH until the composition has a pH in the range of between about 5 and about 5.5.

31. A method of treating a disease or condition in a mammal associated with β₂ adrenergic receptor activity, the method comprising administering to the mammal, a therapeutically effective amount of a pharmaceutical composition of claim 8 or claim 20.

32. The method of claim 31 wherein the disease or condition is a pulmonary disease.

33. The method of claim 32 wherein the pulmonary disease is asthma or chronic obstructive pulmonary disease.

34. The method of claim 31 wherein the disease or condition is selected from the group consisting of pre-term labor, neurological disorders, cardiac disorders, and inflammation.

35. The method of claim 31 wherein the method further comprises administering a therapeutically effective amount of one or more other therapeutic agents.

36. The method of claim 31 wherein the other therapeutic agent is a corticosteroid, an anticholinergic agent, or a PDE4 inhibitor.

37. The method of claim 35 wherein the other therapeutic agent is 6α,9α-difluoro-17α-[(2-furanylcarbonyl)oxy]-11β-hydroxy-16α-methyl-3-oxo-androsta-1,4-diene-17β-carbothioic acid S-fluoromethyl ester or 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester.

38. A process for preparing 2-[4-((R)-2-hydroxy-2-phenylethylamino)phenyl]ethylamine (2): 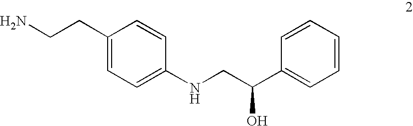
the process comprising the steps of:
(a) reacting 2-(4-aminophenyl)ethylamine or a salt thereof with a sufficient amount of base to substantially deprotonate the 4-amino group; and
(b) reacting the product of step (a) with (R)-styrene oxide to provide compound 2.

39. The process of claim 38, wherein steps (a) and (b) are conducted in a solvent system comprising a polar aprotic solvent.

40. The process of claim 38, wherein the process further comprises forming a crystalline salt of compound 2.
